(19) United States
(12) Patent Application Publication
(10) Pub. No.: US 2018/0303544 A1
(43) Pub. Date: Oct. 25, 2018

(54) SYSTEM, METHOD AND COMPUTER-ACCESSIBLE FOR CATHETER-BASED OPTICAL DETERMINATION OF MET-MYOGLBIN CONTENT FOR ESTIMATING RADIOFREQUENCY ABLATED, CHRONIC LESION FORMATIN IN TISSUE

(71) Applicant: THE TRUSTEES OF COLUMBIA UNIVERSITY IN THE CITY OF NEW YORK, New York, NY (US)

(72) Inventors: Christine P. HENDON, Riverdale, NY (US); Rajinder SINGH-MO, Mastic, NY (US)

(21) Appl. No.: 15/758,639

(22) PCT Filed: Sep. 12, 2016
(86) PCT No.: PCT/US2016/051293

§ 371 Date: Mar. 8, 2018

Related U.S. Application Data
(60) Provisional application No. 62/217,518, filed on Sep. 11, 2015.

(51) Int. Cl. A61B 18/14; A61B 5/00; A61B 5/042

(52) U.S. Cl. CPC A61B 18/1492; A61B 5/0044; A61B 5/0075; A61B 5/14546; A61B 5/0084; A61B 5/0066; A61B 5/0422

(57) ABSTRACT

An exemplary system, method and computer-accessible medium for determining a characteristic(s) of a tissue(s), can be provided which can include, for example, ablating the tissue(s), illuminating the tissue(s) during the ablation procedure; and continuously determining the characteristic(s) based on the ablation and illumination procedures. The tissue(s) can be ablated using radiofrequency ablation. The illumination procedure can be performed with a radiation in a visible spectrum.

### CROSS-REFERENCE TO RELATED APPLICATION(S)

This application relates to and claims priority from U.S. Patent Application No. 62/217,518, filed on Sep. 11, 2015, the entire disclosure of which is incorporated herein by reference.

### STATEMENT REGARDING FEDERALLY SPONSORED RESEARCH

This invention was made with government support under Grant No. HL127776, awarded by the National Institutes of Health, and Grant No. 1454365, awarded by the National Science Foundation. The government has certain rights in this invention.

### FIELD OF THE DISCLOSURE

The present disclosure relates generally to the optical determination of met-myoglobin content, and more specifically, to exemplary embodiments of exemplary system, method and computer-accessible medium for catheter-based optical determination of met-myoglobin content for estimating radiofrequency ablated, chronic lesion formation in tissue (e.g., atrial tissue).

### BACKGROUND INFORMATION

Single-procedure success of radiofrequency ablation (“RFA”) therapies has been largely limited by an inability to characterize lesion sufficiency. Momentarily successful conduction blocks may not be indicative of long-term sustained electrical blockage due to transient effects of edema. (See, e.g., References 1 and 2). Studies have shown that the necrotic lesion core exhibits increased ferric content consistent with a rise in tissue met-myoglobin, as compared to viable tissue. (See, e.g., References 1 and 3).

Thus, it may be beneficial to provide an exemplary system, method and computer-accessible medium which can overcome at least the deficiency described herein above.

### SUMMARY OF EXEMPLARY EMBODIMENTS

An exemplary system, method and computer-accessible medium for determining a characteristic(s) of a tissue(s), can be provided which can include, for example, ablating the tissue(s), illuminating the tissue(s) during the ablation procedure; and continuously determining the characteristic(s) based on the ablation and illumination procedures. The tissue(s) can be ablated using radiofrequency ablation. The illumination procedure can be performed with a radiation in a visible spectrum.

In some exemplary embodiments of the present disclosure, diffuse reflectance spectra can be received based on the illumination procedure, and the characteristic(s) can be determined based on the received diffuse reflectance spectra. The diffuse reflectance spectra can be inverted using an inverse Monte Carlo procedure. In certain exemplary embodiments of the present disclosure, a concentration of an oxy-myoglobin, a deoxy-myoglobin or a met-myoglobin can be determined based on the inverted diffuse reflectance spectra. An exemplary analysis of variance test or a Tukey's multiple comparison test can be performed on the concentration (e.g., the met-myoglobin concentration).

In some exemplary embodiments of the present disclosure, the inverted diffuse reflectance spectra can utilize a wavelength dependent model. A plurality of coefficients can be received based on the fitting, and the characteristic(s) can be determined based on the coefficients. The characteristic(s) can include a classification of the tissue, which can include the tissue(s) having a lesion thereon. The ablation and illumination procedures can be repeated until a permanent lesion is formed on the tissue(s).

In certain exemplary embodiments of the present disclosure, a baseline diffuse reflectance spectra associated with the tissue(s) can be determined before the ablation procedure. In some exemplary embodiments of the present disclosure, the tissue(s) can be flushed and/or the surface of the tissue can be electrically mapped

An exemplary system for determining a characteristic(s) of a tissue(s) can be provided, which can include, for example a first electromagnetic radiation source configured to (i) generate a first radiation(s) and (ii) provide the first radiation(s) to the tissue(s) so as to partially ablate the tissue(s), a second electromagnetic radiation source configured to (i) generate a second radiation(s), and (ii) provide the second radiation(s) to the tissue(s), a detector arrangement configured to (i) obtain a return radiation from the tissue(s) that can be based on the second radiation(s) impacting the tissue(s) and the partial ablation(s) caused by the first radiation(s), and (ii) provide data associated with a further characteristic(s) of the returned radiation, and a computer processing arrangement configured to determine the characteristic(s) based on the data. The data can include information as to whether the tissue(s) has been permanently damaged.

In some exemplary embodiments of the present disclosure, the second radiation can be in a visible spectrum. The characteristic(s) can include a classification of the tissue, which can include the tissue(s) having a lesion thereon. A flushing arrangement(s) can be included, which can be configured to flush the tissue(s). In certain exemplary embodiments of the present disclosure, a voltage arrangement can be included, which can be configured to generate a voltage(s), where the detector arrangement can be further configured to obtain a return voltage from the tissue(s) that can be based on the second radiation(s) impacting the tissue(s). A map(s) of a surface of the tissue(s) can be generated based on the return voltage.

An exemplary method for ablating tissue(s) can be provide, which can include, for example, determining a location(s) of a dead(s) portion of the tissue(s), providing the location(s) to an ablative source arrangement, moving the ablative source arrangement to a further location(s) based on one location(s), and ablating the further location(s) of the tissue(s). The determination procedure can be based on an intensity(ies) and a wavelength(s) of a radiation produced by the ablative source arrangement. In some exemplary embodiments of the present disclosure, the tissue can be flushed using a flushing arrangement and/or the tissue can be mapped using a voltage generator.

An exemplary catheter can be provided, which can include, for example a near infrared radiation generation first arrangement; a visible radiation generating second arrangement, and an ablative arrangement. The catheter can also include a flushing arrangement and/or a voltage generator.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 is a diagram illustrating an exemplary catheter ablating and illuminating tissue according to an exemplary embodiment of the present disclosure;

FIG. 2 is a flow diagram of an exemplary method for classifying tissue according to an exemplary embodiment of the present disclosure;

FIG. 3 is a graph illustrating extinction spectra of dominant chromophores in the visible range in the swine atria according to an exemplary embodiment of the present disclosure;

FIG. 4 is a flow diagram of an exemplary method for radiofrequency ablation according to an exemplary embodiment of the present disclosure;

FIG. 5 is an exemplary diagram illustrating exemplary results for inverting diffuse reflectance measurements to RF parameters according to an exemplary embodiment of the present disclosure;

FIG. 6 is a set of graphs illustrating exemplary representative spectra and corresponding fitting results for three exemplary groups: untreated (e.g., top row), mildly treated (e.g., middle row), moderately treated (e.g., bottom row) according to an exemplary embodiment of the present disclosure;

FIG. 7 is a graph illustrating exemplary statistical comparison of optically determined Mmb concentrations for varying degrees of RF treated tissue according to an exemplary embodiment of the present disclosure;

FIG. 8 is a set of graphs illustrating reflectance spectra, extracted absorption and scattering spectra from visible light spectroscopy according to an exemplary embodiment of the present disclosure;

FIG. 9 is a set of graphs illustrating examples of real-time monitoring of RF ablation according to an exemplary embodiment of the present disclosure;

FIG. 10 is a set of graphs illustrating the effect of RF treatment on optically determined met-myoglobin concentration according to an exemplary embodiment of the present disclosure;

FIG. 11 is a diagram of an exemplary catheter according to an exemplary embodiment of the present disclosure;

FIG. 12 is a diagram illustrating an exemplary catheter ablating and illuminating tissue according to an exemplary embodiment of the present disclosure;

FIG. 13 is a flow diagram of an exemplary method for treating a lesion according to an exemplary embodiment of the present disclosure;

FIG. 14 is an exemplary diagram of the exemplary catheter according to an exemplary embodiment of the present disclosure;

FIG. 15A is a diagram illustrating a source-detector separation according to an exemplary embodiment of the present disclosure;

FIG. 15B is a set of maps illustrating the source-detector separation on measured signals according to an exemplary embodiment of the present disclosure;

FIG. 16 is a set of maps illustrating the impact of tissue absorption and scattering parameters on the measured reflectance for various source-detector separations according to an exemplary embodiment of the present disclosure;

FIG. 17 is a set of diagrams of exemplary catheter sheaths according to an exemplary embodiment of the present disclosure;

FIG. 18 is a diagram of an optically-integrated mapping catheter according to an exemplary embodiment of the present disclosure;

FIG. 19 is a set of images illustrating ex-vivo lesion mapping setup according to an exemplary embodiment of the present disclosure;

FIG. 20 is a set of exemplary optical parameter maps of the swine right ventricular lesion set produced using the exemplary catheter according to an exemplary embodiment of the present disclosure;

FIG. 21 A is an exemplary fluoroscopy image produced using the exemplary catheter according to an exemplary embodiment of the present disclosure;

FIG. 21B is an exemplary graph illustrating extracted metmyoglobin dynamics during a 60 second cardiac ablation according to an exemplary embodiment of the present disclosure;

FIG. 21C is an exemplary image of a resulting lesion after tetrazolium chloride staining according to an exemplary embodiment of the present disclosure;

FIG. 22A is a flow diagram of an exemplary method for determining a characteristic of a tissue according to an exemplary embodiment of the present disclosure;

FIG. 22B is a flow diagram of an exemplary method for ablating a tissue according to an exemplary embodiment of the present disclosure; and

FIG. 23 is an illustration of an exemplary block diagram of an exemplary system in accordance with certain exemplary embodiments of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXEMPLARY EMBODIMENTS

### Exemplary Method

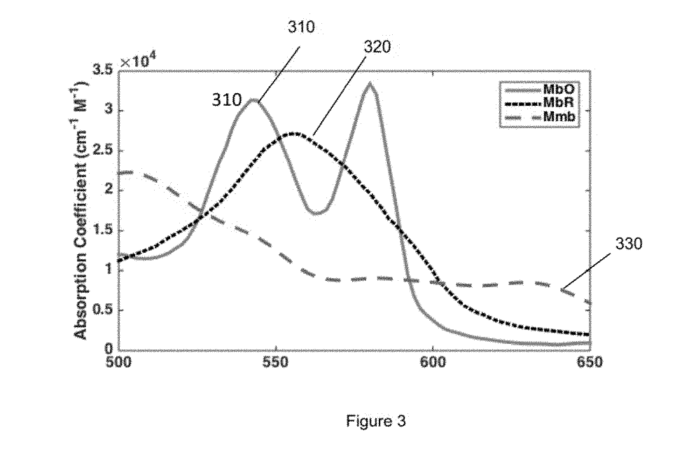

According to one exemplary embodiment of the present disclosure, a fiber-optic integrated RFA catheter was used to obtain broadband (e.g., 500-650 nm) diffuse reflectance measurements at a source-detector separation of 0.8 mm at the catheter tip. Atrial samples were excised from two fresh swine hearts and supraperfused in warm (e.g., 37° C.) phosphate buffered saline. Optical measurements were taken for three RFA-treated tissue groups: untreated (e.g., n=7), mildly treated (e.g., n=3), and moderately treated (e.g., n=4). An inverse Monte Carlo procedure was used to invert diffuse reflectance spectra to recover concentrations of oxy-myoglobin (“MbO”), deoxy-myoglobin (“Mb”), and met-myoglobin (“Mmb”). Comparisons across the groups revealed significantly greater Mmb concentrations (e.g., p<0.0001) in the moderately treated group as compared to the other two. Additionally, an increasing trend in Mmb concentration was observed for increased tissue treatment. Absorption contributions to the measured signal was modeled as a weighted sum of MbO, Mb, and Mmb extinction spectra c (e.g., FIG. 3) as shown in the expression below:

μₐ(λ)=c_{mbO}·ε_{mbO}(λ)+c_{Mb}·ε_{Mb}(λ)+c_{Mmb}·ε_{Mmb}(λ) (1)

where c can be the chromophore concentration. Reduced scattering was modeled as a weighted sum of Rayleigh and Mie scatterer as described below:

$${μ}_{s}^{′} ( λ ) = {a ( \frac{λ}{600 nm} )}^{- 4} + {b ( \frac{λ}{600 nm} )}^{- d} . \qquad ( 2 )$$

An analysis of variance (“ANOVA”), along with Tukey's multiple comparison test, were performed for the extracted concentrations of Mmb across the groups. A p-value of 0.5 was used to denote significance.

An optically-integrated catheter was used to measure three groups of RFA-treated, swine atria: untreated, mildly treated, and moderately treated. Concentrations for oxy-myoglobin, deoxy-myoglobin and met-myoglobin were determined using an inverse Monte Carlo scheme. Met-myoglobin concentrations were significantly greater (e.g., p<0.0001) for the moderately treated group compared to the others groups.

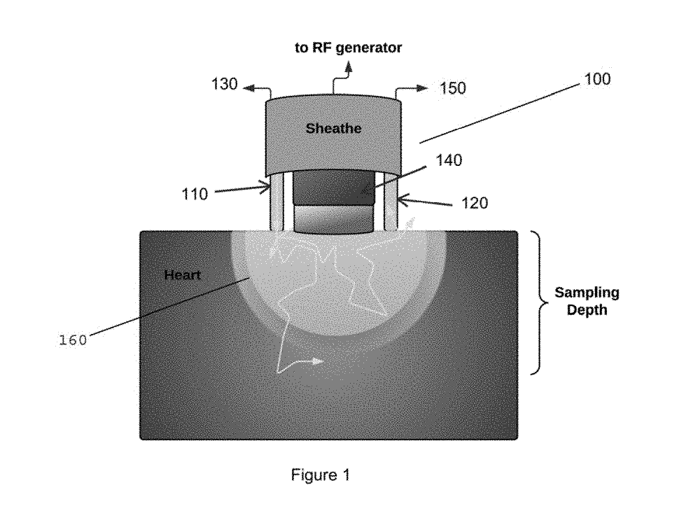

FIG. 1 illustrates an exemplary system setup 100 for obtaining measurements from myocardial tissues as well as the zone of resistive heating during ablation. Optical fibers can be embedded in a sheathe whose inner channel accepts an ordinary commercial RFA catheter 140. Two sets of fibers can typically be employed: illumination fiber(s) 110 and collection fibers 120. Broadband light 130 can be delivered onto the heart 160 via the illumination fiber(s). The tissue diffusely backscattered light can then be recovered by the collection fiber(s) 120 placed at some distance away from the illumination point and recorded by a spectrometer 150. Collected photons can sample the myocardium and can contain information on physiological makeup and ultrastructure of the traverse path. Fibers can be integrated into the sheath or into the ablation catheter.

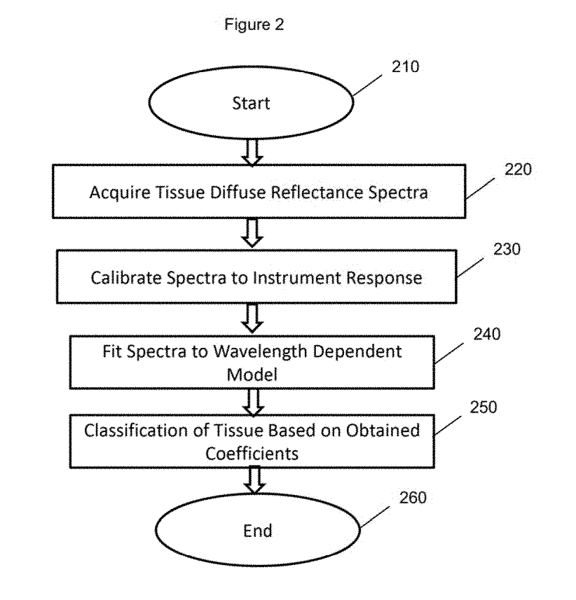

FIG. 2 shows a flow diagram of an exemplary method for tissue classification according to an exemplary embodiment of the present disclosure. For example, at procedure 210, the exemplary method can begin, or can run continuously as initiated by a computer or a user. At procedure 220, a diffuse reflectance spectra can be acquired. At procedure 230, a calibration procedure can be carried out with respect to reference standards. At procedure 240, a wavelength dependent model can be used to fit to the reflectance spectra. Subsequent properties can be derived from the model, such as absorption spectra, scattering spectra and chromophore composition. At procedure 250, using the properties/features extracted from the reflectance spectra using the wavelength dependent model, classification of the tissue can be performed. This can include identification of fat, fibrous tissue, collagen, normal myocardium, infarction, chronic ablation lesion or acute ablation lesion. At procedure 260, the exemplary procedure can be run continuously, classifying tissue until the procedure can be over or until the user stops the processing.

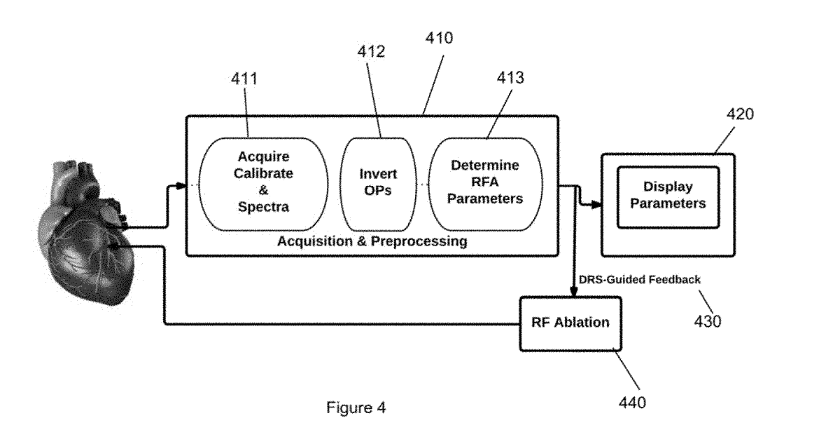

As illustrates in the graph shown in FIG. 3, Extinction spectra for dominant chromophores in the visible range can be used for fitting cardiac tissue. Spectra were derived from equine heart (e.g., MbO-oxymyoglobin 310, Mb-deoxymyoglobin 320, Mmb-metmyoglobin 330). Protocol for real-time guidance of RF ablation procedures (e.g., shown in the flow diagram of FIG. 4) can include an acquisition and pre-processing of reflectance spectra procedure 410. The acquisition and pre-processing of reflectance spectra procedure can include, e.g., the acquisition of the calibration information and the spectra at procedure 411, the inversion of the Ops at procedure 412 and the determination of the RFA parameters at procedure 413. The parameters and the tissue classification can be displayed at procedure 420, with the parameters being used to guide feedback at procedure 430, including titrating ablation power, intensity, temperature, or dose. The adjustment of ablation source energy parameters can be performed at procedure 440 until the desired parameters (e.g., lesion depth) can be achieved.

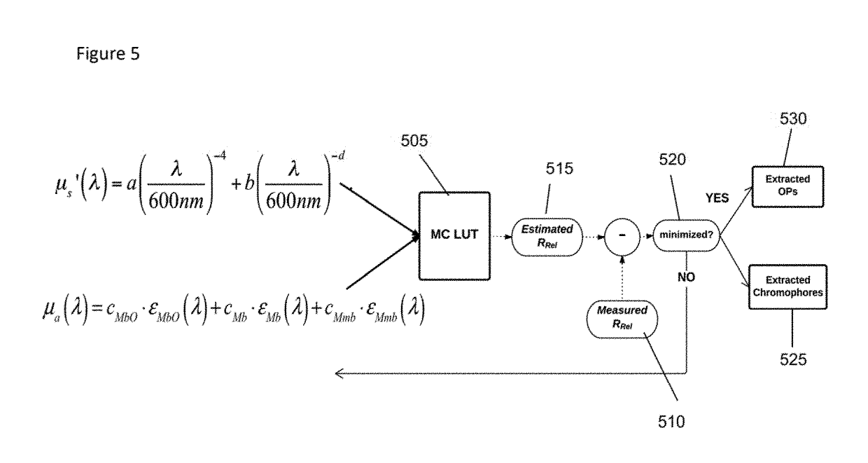

FIG. 5 illustrates exemplary results of inverting Diffuse Reflectance measurements to RF parameters according to an exemplary embodiment of the present disclosure. A precomputed look-up table 505 can be used as a forward model that takes in a combination of absorption and reduced scattering coefficients and outputs the diffuse reflectance for the illumination-collection geometry. For example, the absorption profile can be modeled as a weighted sum of dominant chromophores oxy-myoglobin, deoxy-myoglobin and met-myoglobin. Reduced scattering spectra can be modeled as a sum of Rayleigh and Mie scatterers. The error between the measured relative reflectance (“Rrel”) 510 and the predicted (e.g., estimated) Rrel 515 can be minimized 520 or otherwise reduce in the least-squares sense by finding the optimal coefficient values within the absorption and reduced scattering expressions. The extracted chromophores 525 and optical properties 530 could then be used for tissue substrate determination and inferring lesion characteristics.

### Exemplary Results

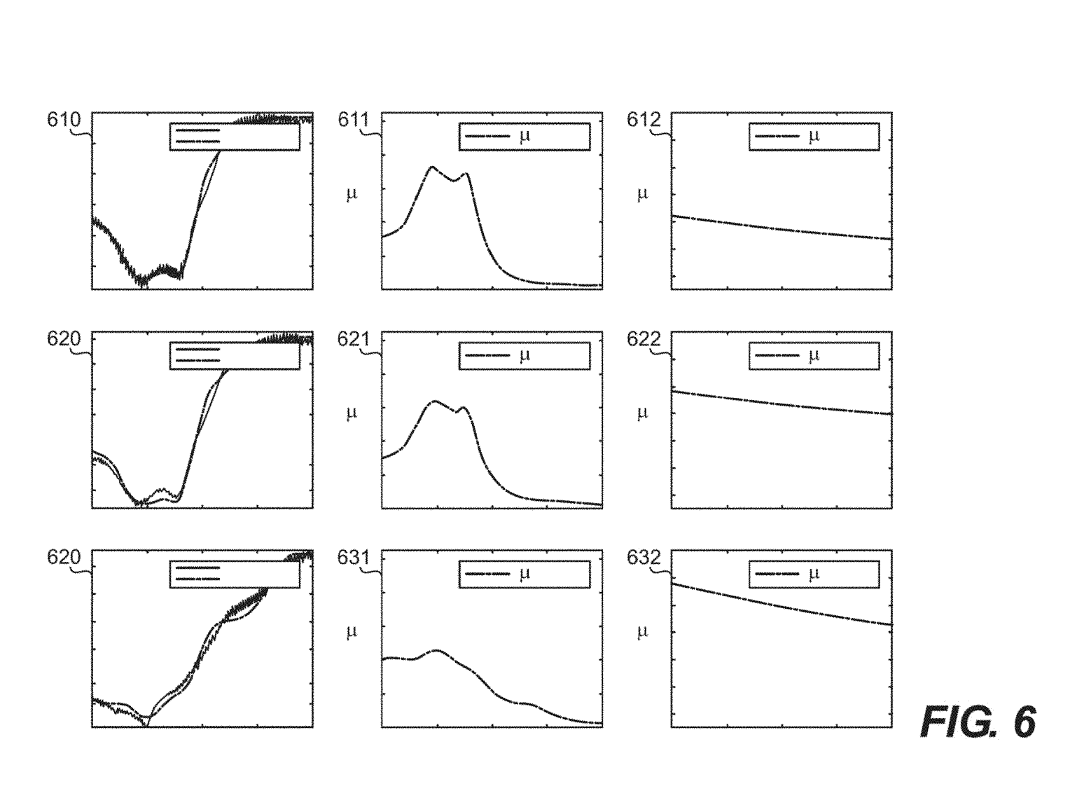

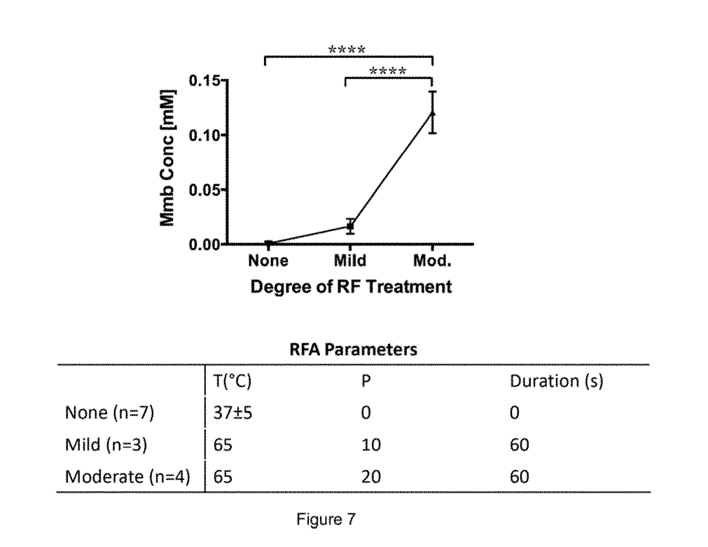

FIG. 6 shows a set of exemplary graphs of representative optical measurements for three group according to an exemplary embodiment of the present disclosure. For example, the exemplary graphs shown in FIG. 6 illustrate the effect of radiofrequency ablation on measurements in ex vivo right atrium samples (e.g., swine). For example, graphs 610, 611 and 612 show representative spectral fitting and optical property extraction for the untreated myocardium. Graphs 620, 621 and 622 and graphs 630, 631 and 632 indicate similar measurements obtained for the light treated myocardium (e.g., graphs 620, 621 and 622) and moderately treated myocardium (e.g., graphs 630, 631 and 632) moderately treated myocardium. An increase in scattering can be observed with increasing lesion depth. In addition, the spectral shape of the absorption curve changes with moderate treatment, compared with untreated and lightly treated. FIG. 7 shows a chart illustrating that the comparisons across the groups revealed significantly greater Mmb concentrations (e.g., p<0.0001) in the moderately treated group as compared to the other two groups (e.g., the effect of RF treatment on optically determined tissue met-myoglobin concentration in the swine right atrium). Additionally, an increasing trend in Mmb concentration was observed for increased tissue treatment (e.g., see graph shown in FIG. 7).

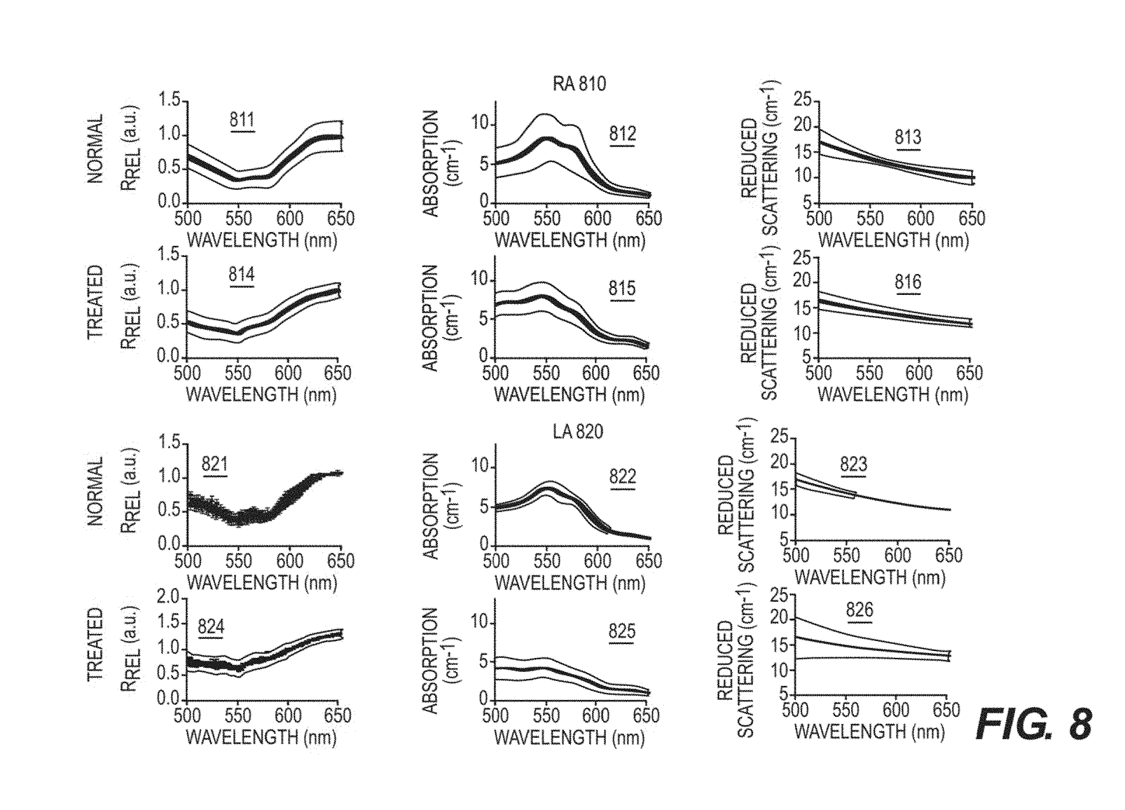

FIG. 8 shows a set of exemplary graphs illustrating examples of reflectance spectra (e.g., graphs 811, 814, 821 and 824), extracted absorption (e.g., graphs 812, 815, 822 and 825) and scattering spectra (e.g., graphs 813, 816, 823, 826) from visible light spectroscopy. Mean and standard deviations for optical measurements in right atrium (e.g., RA 810) and left atrium (e.g., LA 820) from four swine hearts, for normal untreated areas and areas treated with radiofrequency ablation. The change in absorption spectra morphology for treated tissue (e.g., graphs 815 and 825) suggests a difference in biochemical composition compared to untreated tissue.

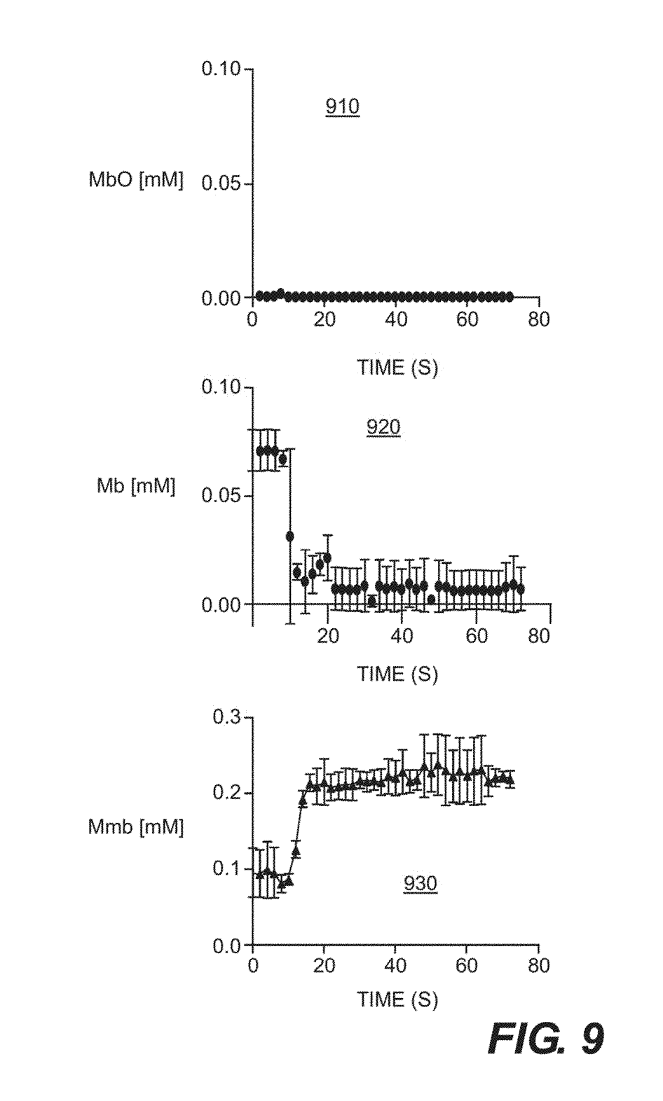

FIG. 9 illustrates a set of graphs illustrating examples of real-time monitoring of RF ablation in human atrial tissue with visible light spectroscopy. Ablation started at t=8s. For example, graph 920 shows a decrease in deoxy-myoglobin can be observed from the onset of RF ablation. Graph 930 shows a corresponding increase in met-myoglobin content can be observed as RF energy can be continually applied to the tissue. Graph 910 shows concentrations of oxy-myoglobin were negligible and did not change significantly during ablation.

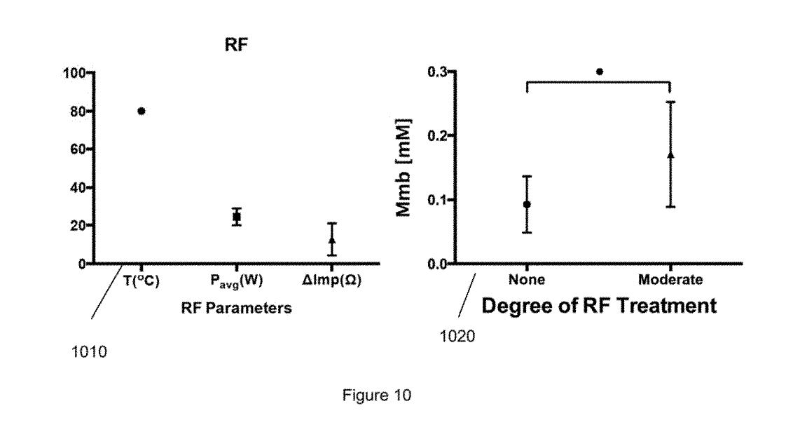

FIG. 10 shows a set of graphs illustrating examples of the affect or RF treatment on optically determined met-myoglobin concentration in an ex-vivo human right atrium. For example, graph 1010 shows the standard parameters measured from the ablation catheter, temperature (“T”), Average Power (“Pavg”) and change in impedance (e.g., Δimp(Ω)). Met-myoglobin concentration significantly increased between untreated and moderately treated lesions, (e.g., P<0.05).

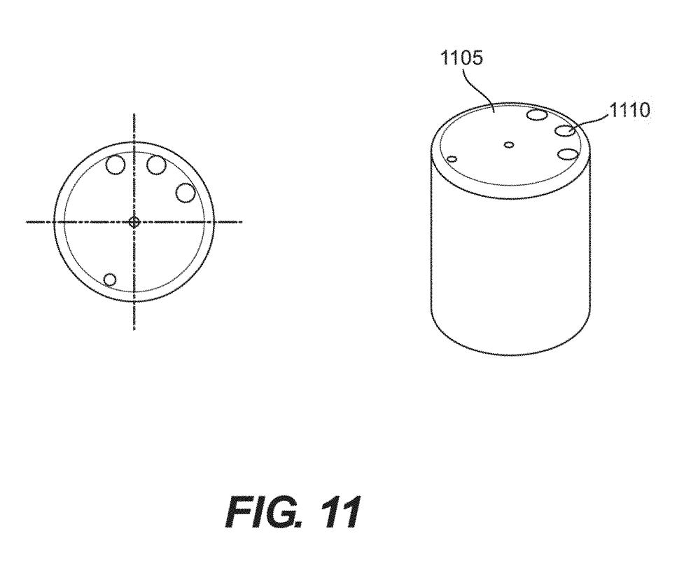

FIG. 11 shows a diagram of an exemplary design for a catheter tip 1105 containing slots 1110 for multiple optical fibers according to an exemplary embodiment of the present disclosure. Sensitivities to absorption and scattering can vary with source detector separation. Thus, full-spectrum reflectance data can be leveraged by simultaneously acquiring data from multiple distances away from the source. Furthermore, multiple source-detector pairs can be used to determine relative contact angle of catheter to tissue surface. Collection fibers can be used for visible light spectroscopy, near infrared spectroscopy, autofluorescence or optical coherence tomography.

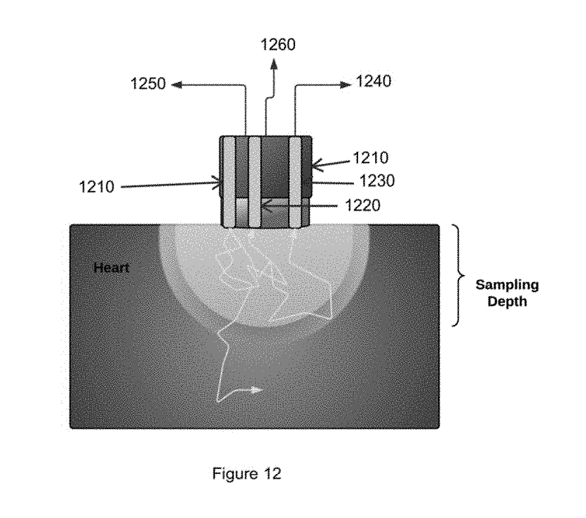

As shown in the diagram of FIG. 12, a fiber-integrated catheter 1200 can be used to combine visible and NIR spectral measurements. The target tissue sample can be illuminated with a source energy 1250 with an illumination fiber 1210. The close source-detector fiber separations (“SDFS”) can probe light that does not travel very deep into the tissue (e.g., using close collection fiber 1220). The wide SDFS 1230 can measure light that can probe deeper into the tissue and can be more sensitive tissue absorption. Because the absorption of dominant metalloproteins in cardiac tissue can be orders of magnitude higher in the visible region than the NIR, close SDFS can be used to measure the apparent large absorption in the visible region while using wide SDFS collection to appreciate the more modest absorption changes in the NIR regime. Both light collected through close collection fiber 1220 and wide SDFS 1230 can return to the spectrometer 1240 for detection. Reflectance spectra from both detection fibers can enable tissue characterization, chromophore composition analysis, and contact angle determination during the process of the ablation procedure for pre-procedural substrate classification and real time evaluation during the application of ablation energy 1260.

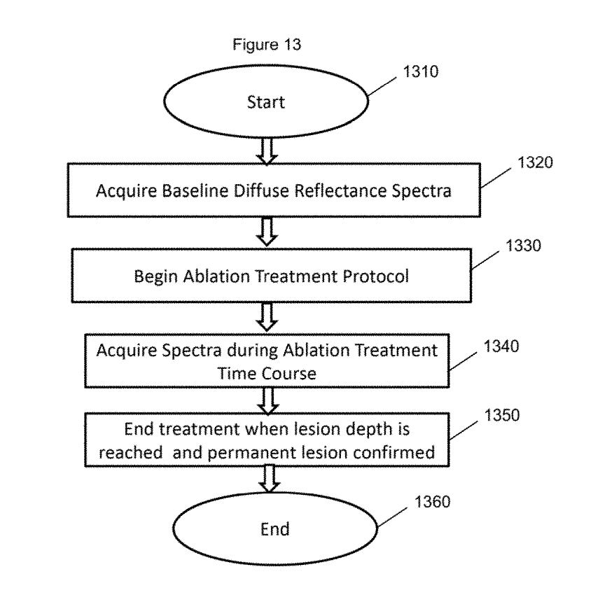

FIG. 13 shows a flow diagram of an exemplary method for lesion depth monitoring according to an exemplary embodiment of the present disclosure. Visible light spectroscopy can aid in verifying that the lesion produced in permanent and NIR spectroscopy can verify lesion depth. For example, at procedure 1310 the exemplary method can be run continuously or initiated by user. At procedure 1320, diffuse reflectance spectra can be acquired. At procedure 1330, ablation treatment can begin by turning on source energy, such as radiofrequency, laser, ultrasound or cryo. At procedure 1340, real-time spectra can be acquired during the ablation treatment time course. A wavelength dependent model can be used to fit to the reflectance spectra. Subsequent properties can be derived from the model, such as absorption spectra, scattering spectra and chromophore composition. At procedure 1350, using the properties/features extracted from the reflectance spectra using the wavelength dependent model, classification of the tissue can be performed. Status of the ablation lesion formation can be provided, included proxies for ablation lesion depth, whether permanent injury has occurred, whether a steam pop has occurred, and whether contact is being maintained with the tissue. At procedure 1360, the exemplary method can run continuously, classifying tissue until the procedure can be over when the target lesion depth has been achieved.

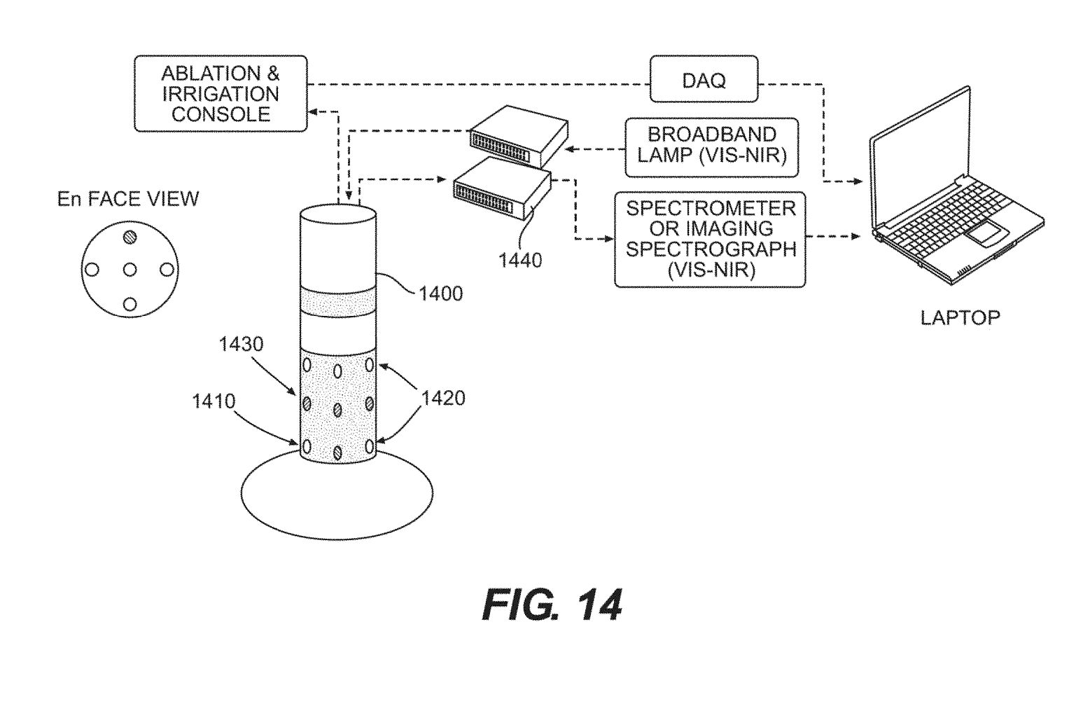

FIG. 14 shows an exemplary diagram of the exemplary catheter according to an exemplary embodiment of the present disclosure. For example, as shown in the diagram of FIG. 14, the exemplary catheter 1400 can be used for optical fiber-integrated radiofrequency ablation. The exemplary catheter 1400 can include one or more electrode tip houses 1410 illumination and one or more sets of collection optodes 1420, as well as openings 1430 for a saline irrigation/flushing. Both fiber can be are distributed along the circumference and face of the metal tip of catheter 1400. High speed 1440 fiber optic switches can be used to alternate between source and detector positions. The contact orientation with the tissue surface can be estimated using catheter 1400 by multi-detector measurements for all possible given source positions. The tissue characteristics can then be determined by fitting an exemplary light transport model to the geometry spanned by the subset of optodes that are in contact.

Further, the illumination location can be alternated while stimulation of the tip side can be distributed throughout the arranged to position, which can be alternatingly scanned throughout any given sets of holes. The exemplary catheter 1400 can be used for obtaining measurements from myocardial tissues as well as the zone of resistive heating during ablation. Optical fibers can be in a sheathe where an inner channel can accept a commercially available RFA catheter. Two sets of fibers can typically be employed (e.g., illumination and collection). Broadband light can be delivered onto the heart via one or more of the illumination fiber. The tissue can be diffusely backscattered light, which can then be recovered by the collection fibers, which can be placed at some distance away from the illumination point. Collected photons samples of the myocardium can contain information on physiological makeup and ultrastructure of the traverse path.

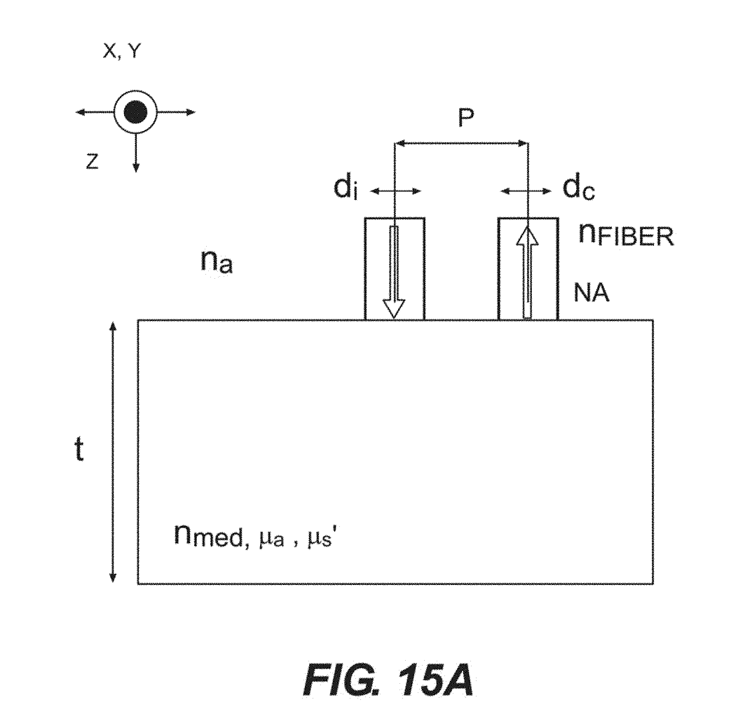

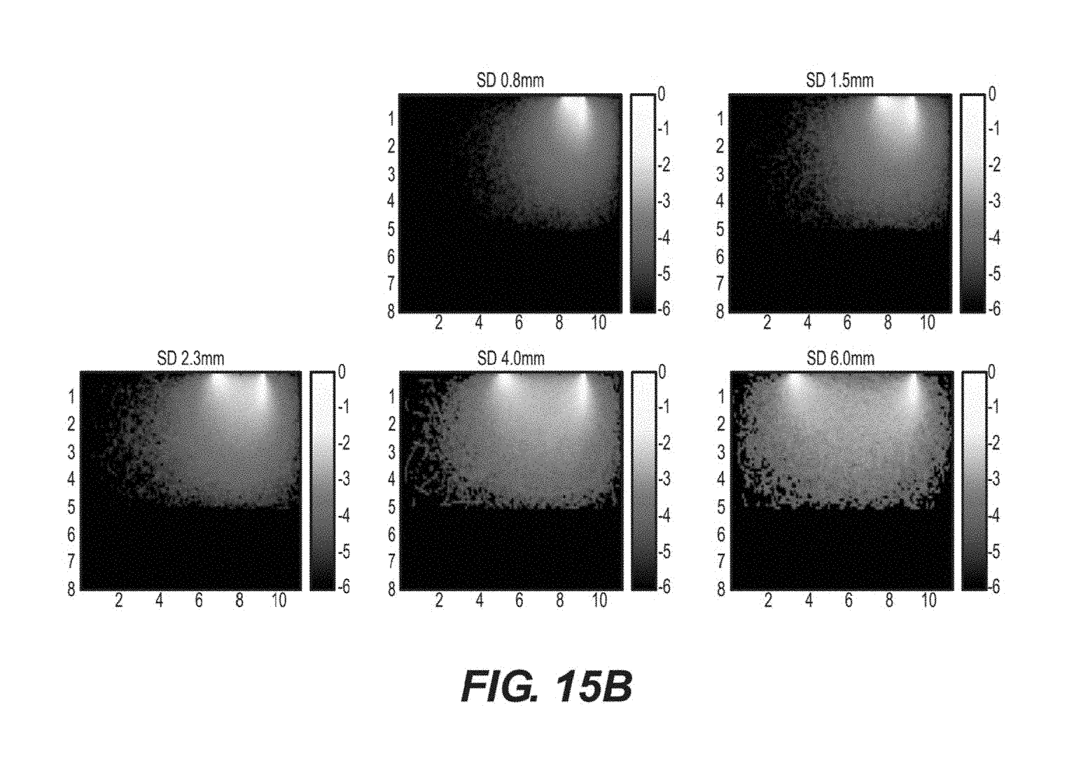

FIG. 15A shows a diagram illustrating source-detector separation and FIG. 15B shows a set of charts illustrating the impact of source-detector separation on measured signals according to an exemplary embodiment of the present disclosure. Monte Carlo simulations were performed for various illumination-collection geometries for a 5 mm thick slab with a fixed optical properties. (See, e.g., FIG. 15B). The Jacobian shows a greater depth of tissue interrogated with increased fiber separations.

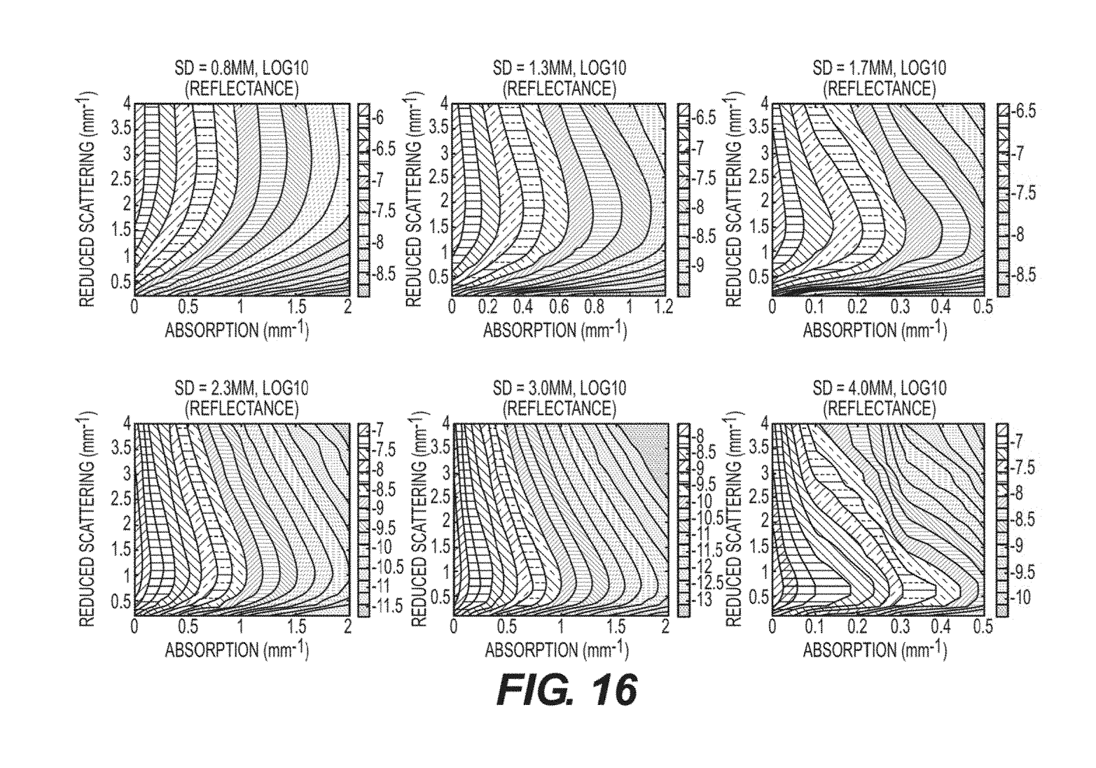

FIG. 16 shows a set of exemplary maps illustrating the impact of tissue absorption and scattering parameters on measured reflectance for various source-detector separations according to an exemplary embodiment of the present disclosure. For example, as shown in FIG. 16, for smaller fiber separations, increases in reduced scattering generally results in increased signal intensity. For larger separations, a momentary increase is shows, followed by a gradually decrease in the signal. The increased separation alters the scattering value at which this inflection point occurs, as well as the rate of decrease due to scattering. Additionally, larger source detector separations experience greater sensitivities to absorption due to the longer path length traveled by collected photons.

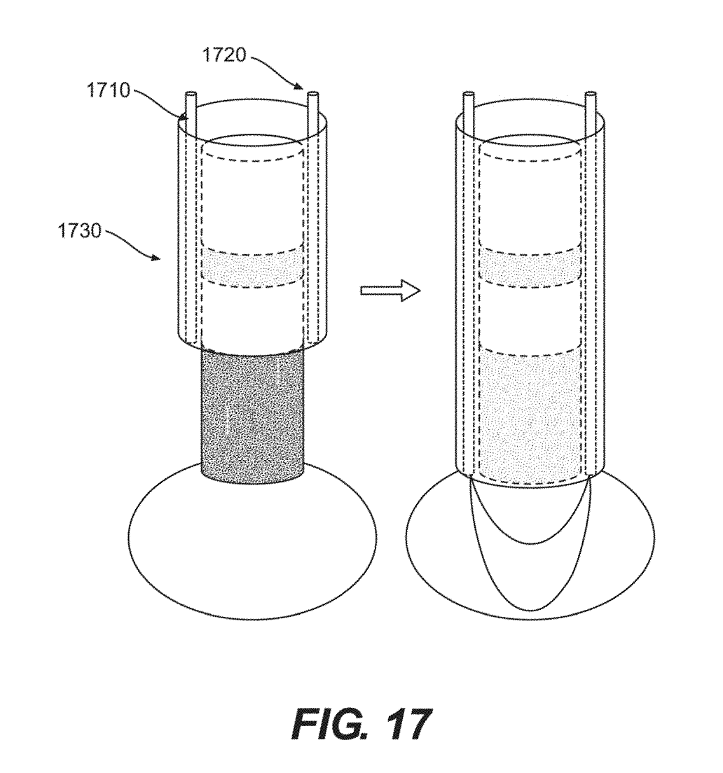

FIG. 17 shows a set of diagrams of exemplary catheter sheaths according to an exemplary embodiment of the present disclosure. The exemplary sheaths can be optically-integrated, and can utilize the sheathes for tissue characterization during the exemplary RF procedures. Illumination fibers 1710 and collection fibers 1720 can be placed along the sheathe wall 1730. An insertion through the inner channel can facilitate supplemental optical measurements to be taken using any commercial catheter.

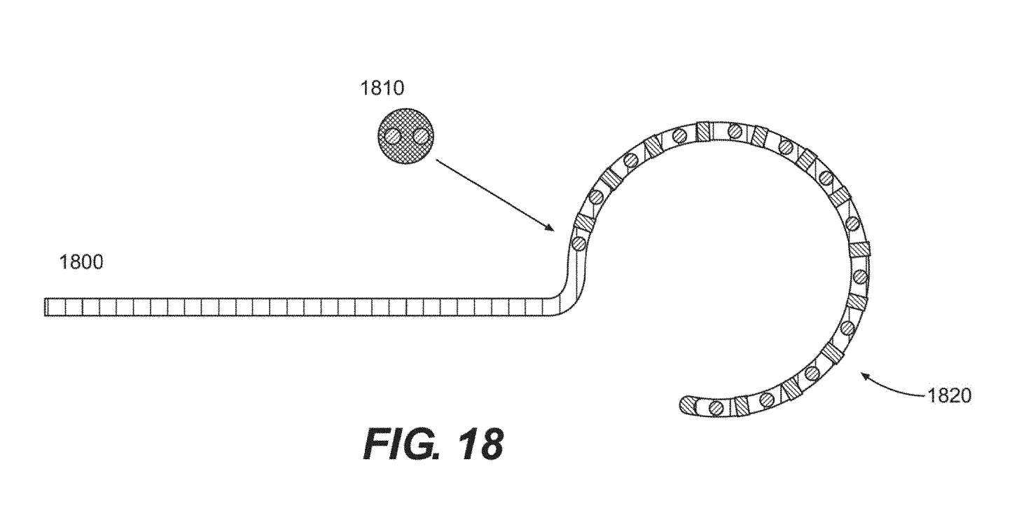

FIG. 18 shows a diagram of an optically-integrated mapping catheter 1800 according to an exemplary embodiment of the present disclosure. For example, as shown in FIG. 18, illumination-collection pairs 1810 can be placed alongside electrodes 1820, to facilitate simultaneous electrical measurements along with local optical tissue characterization. Optical parameters point clouds, or surface maps, can be generated using positional information provided by navigational systems and can provide information on lesion gaps and lesion inadequacy.

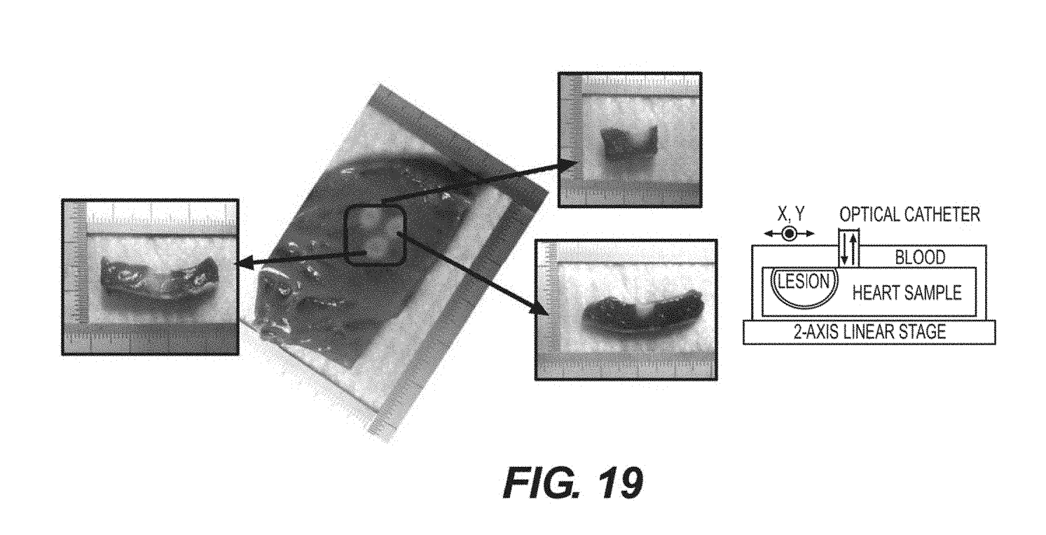

FIG. 19 shows a set of images illustrating ex-vivo lesion mapping setup according to an exemplary embodiment of the present disclosure. A swine right ventricular wedge was ablated with various lesion sizes. The sample was submerged in blood, and optical measurements were made across the surface. The catheter was translated using a two-axis linear stage, which provided spatial coordinates for optical parameters maps. Lesion depth was determined using an approximately 1% tetrazolium chloride staining post-optical measurements.

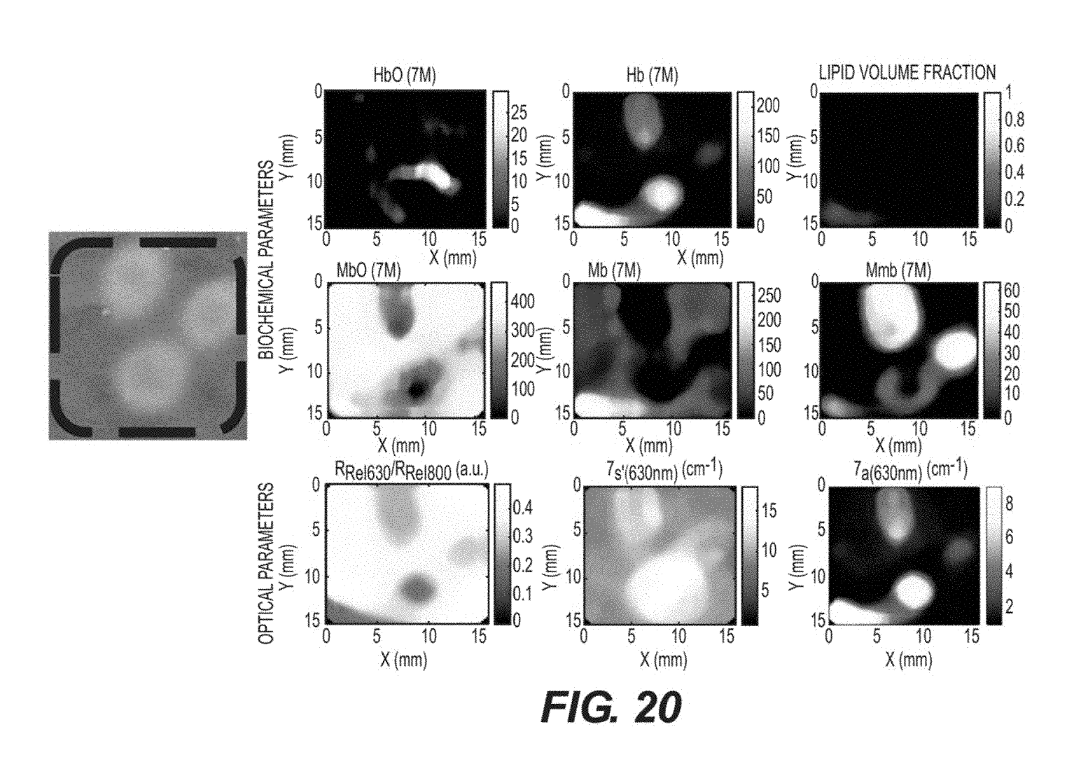

FIG. 20 shows a set of optical parameter maps of the swine right ventricular lesion set produced using the exemplary catheter according to an exemplary embodiment of the present disclosure. For example, the Metmyoglobin maps shown in FIG. 20 illustrate particular sensitivity to treated sites in biochemical maps. Non-specific signals can be a result of cross-talk effects due to the high absorption in blood pools where the catheter may not be in full contact. Optical parameters show strong concordance with the extent of treatment. Additionally, the scattering maps are relatively insensitive to blood pooling sites.

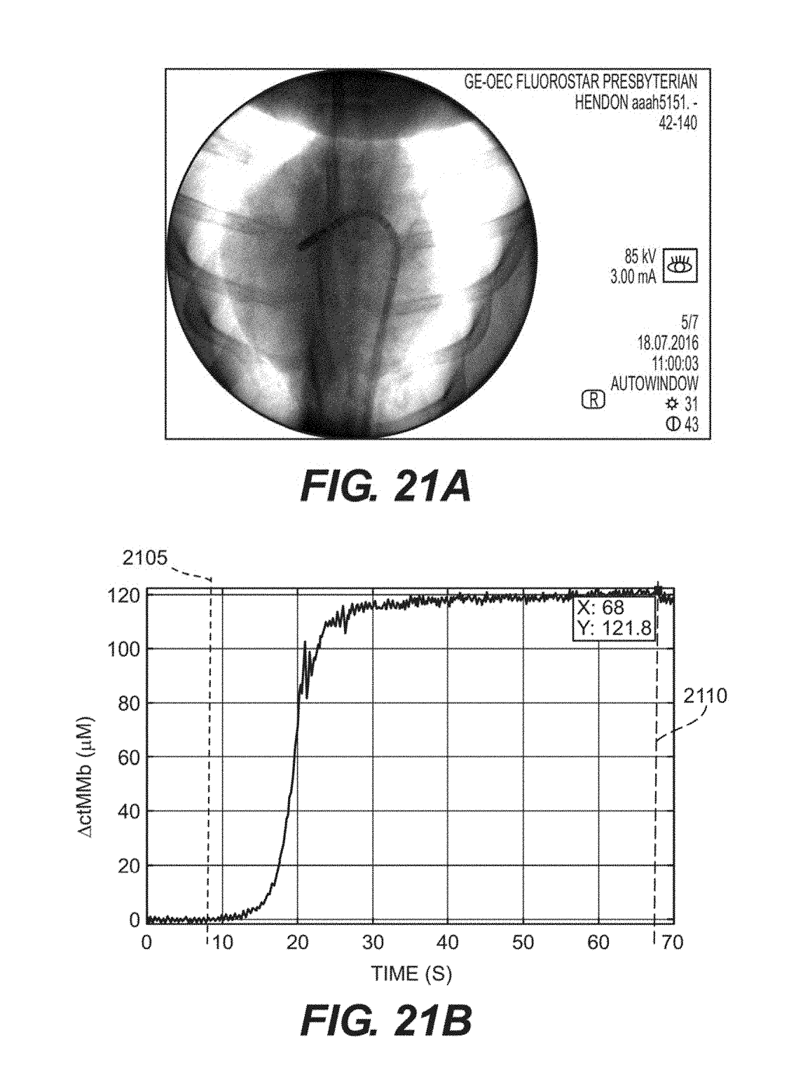

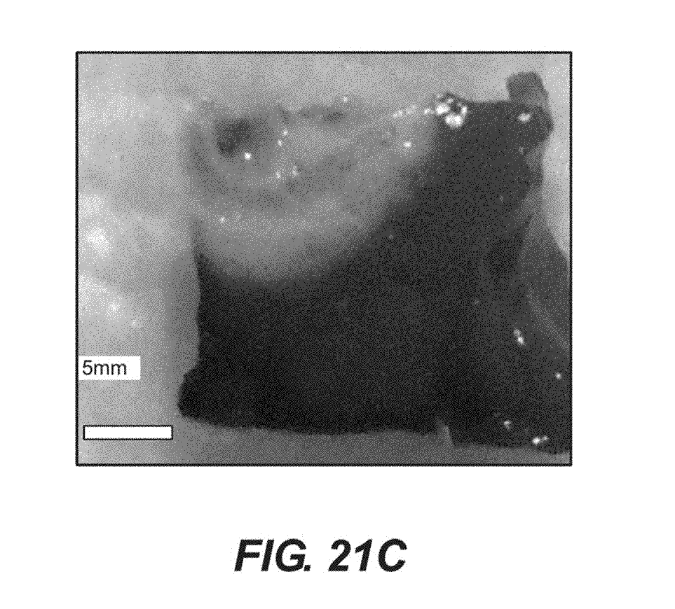

FIG. 21A shows an exemplary fluoroscopy image of the exemplary catheter being steered within the heart produced using the exemplary catheter according to an exemplary embodiment of the present disclosure. FIG. 21B shows an exemplary graph illustrating extracted metmyoglobin dynamics during a 60 second cardiac ablation according to an exemplary embodiment of the present disclosure. RF initiation is marked by the dashed line 2105 while RF termination is marked by the dashed line 2110. FIG. 21C shows an exemplary image of a resulting lesion after tetrazolium chloride staining according to an exemplary embodiment of the present disclosure.

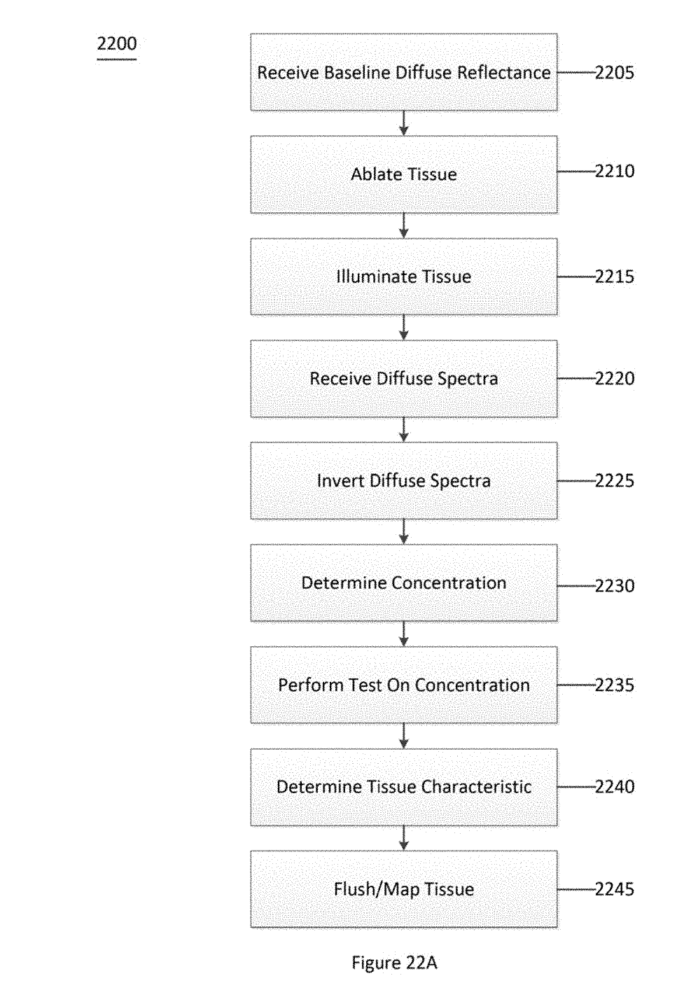

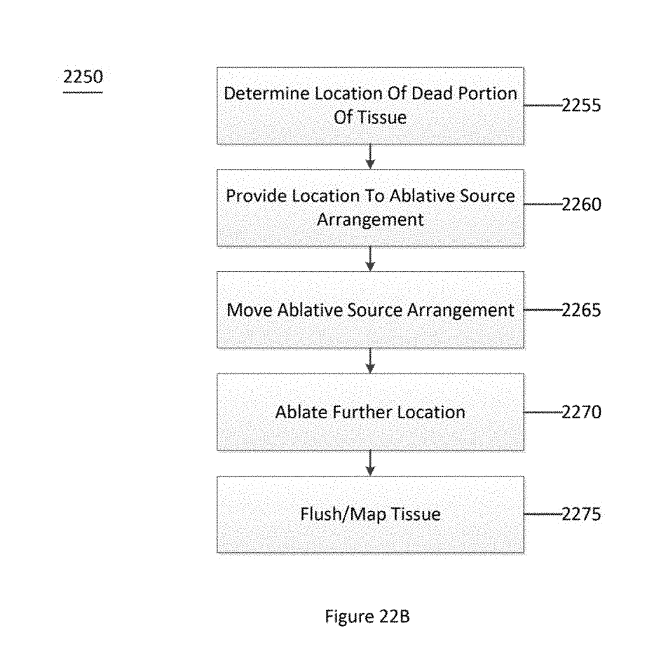

FIG. 22A shows a flow diagram of an exemplary method 2200 for determining a characteristic of a tissue according to an exemplary embodiment of the present disclosure. For example, at procedure 2205, a baseline diffuse reflectance spectra of the tissue can be received. At procedure 2210, the tissue can be ablated, and at procedure 2215, the tissue can be illuminated. At procedure 2220, diffuse reflectance spectra can be received based on the illumination from procedure 2215, which can be inverted at procedure 2225. At procedure 2230, a concentration (e.g., oxy-myoglobin, a deoxy-myoglobin and a met-myoglobin) can be determines based on the inverted diffuse reflectance spectra. At procedure 2235, a test (e.g., an analysis of variance test or Tukey's multiple comparison test) can be performed on the met-myoglobin. At procedure 2240, a characteristic of the tissue can be determined. Additionally, at procedure 2245, the tissue can be flushed, or the tissue can be mapped (e.g., using voltage mapping).

FIG. 22B shows a flow diagram of an exemplary method 2250 for ablating a tissue according to an exemplary embodiment of the present disclosure. For exemplary, at procedure 2255, a location of a dead portion of a tissue can be determined. This location can be provided to an ablative source arrangement at procedure 2260. At procedure 2265, the ablative source arrangement can be moved to a further location, and the further location can be ablated at procedure 2270. Additionally, at procedure 2275, the tissue can be flushed, or the tissue can be mapped (e.g., using voltage mapping).

### DISCUSSION AND CONCLUSION

Exemplary results indicate that met-myoglobin quantification can serve as an important marker for estimating increased tissue treatment. Furthermore, these measurements can be facilitated by real-time optical measurements made at the RFA catheter tip.

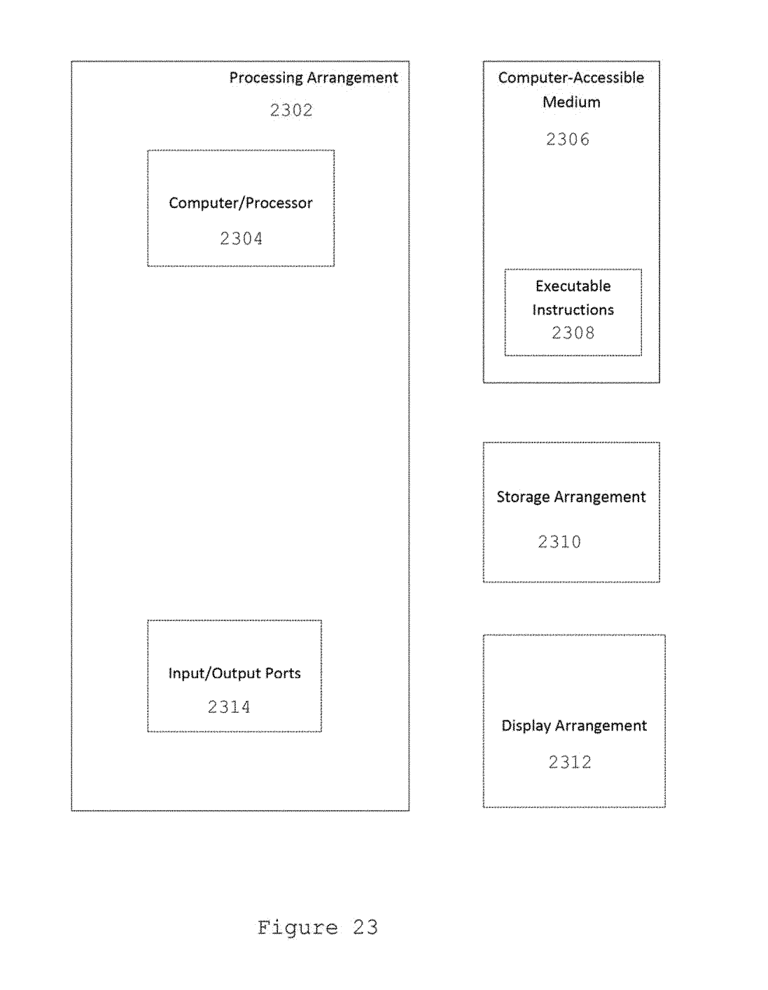

FIG. 23 shows a block diagram of an exemplary embodiment of a system according to the present disclosure. For example, exemplary procedures in accordance with the present disclosure described herein can be performed by a processing arrangement and/or a computing arrangement 2302. Such processing/computing arrangement 2302 can be, for example entirely or a part of, or include, but not limited to, a computer/processor 2304 that can include, for example one or more microprocessors, and use instructions stored on a computer-accessible medium (e.g., RAM, ROM, hard drive, or other storage device).

As shown in FIG. 23, for example a computer-accessible medium 2306 (e.g., as described herein above, a storage device such as a hard disk, floppy disk, memory stick, CD-ROM, RAM, ROM, etc., or a collection thereof) can be provided (e.g., in communication with the processing arrangement 2302). The computer-accessible medium 2306 can contain executable instructions 2308 thereon. In addition or alternatively, a storage arrangement 2310 can be provided separately from the computer-accessible medium 2306, which can provide the instructions to the processing arrangement 2302 so as to configure the processing arrangement to execute certain exemplary procedures, processes and methods, as described herein above, for example.

Further, the exemplary processing arrangement 2302 can be provided with or include an input/output arrangement 2314, which can include, for example a wired network, a wireless network, the internet, an intranet, a data collection probe, a sensor, etc. As shown in FIG. 23, the exemplary processing arrangement 2302 can be in communication with an exemplary display arrangement 2312, which, according to certain exemplary embodiments of the present disclosure, can be a touch-screen configured for inputting information to the processing arrangement in addition to outputting information from the processing arrangement, for example. Further, the exemplary display 2312 and/or a storage arrangement 2310 can be used to display and/or store data in a user-accessible format and/or user-readable format.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### EXEMPLARY REFERENCES

The following references are hereby incorporated by reference in their entirety.
1. H. Celik, V. Ramanan, J. Barry, S. Ghate, V. Leber, S. Oduneye, Y. Gu, M. Jamali, N. Ghugre, J. A. Stainsby, M. Shurrab, E. Crystal, and G. A. Wright, “Intrinsic contrast for characterization of acute radiofrequency ablation lesions,” Circulation. Arrhythmia and electrophysiology 7, 718-727 (2014).
2. D. P. Zipes and J. Jalife, Cardiac electrophysiology: from cell to bedside, Sixth edition. ed. (Elsevier/Saunders, Philadelphia, Pa., 2014), pp. xxvi, 1365 pages.
3. J. Swartling, S. Palsson, P. Platonov, S. B. Olsson, and S. Andersson-Engels, “Changes in tissue optical properties due to radio-frequency ablation of myocardium,” Medical & biological engineering & computing 41, 403-409 (2003).
4. R. M. Singh-Moon, C.; Hendon, C., “Near-infrared spectroscopy integrated catheter for characterization of myocardial tissues: preliminary demonstrations to radiofrequency ablation therapy for atrial fibrillation,” Biomed. Opt. Express 6, 2494-2511 (2015).

1. A method for determining at least one characteristic of at least one tissue, comprising:
ablating the at least one tissue;
illuminating the at least one tissue during the ablation procedure; and
using a computer hardware arrangement, continuously determining the at least one characteristic based on the ablation and illumination procedures.

2. The method of claim 1, further comprising ablating the at least one tissue using radiofrequency ablation.

3. The method of claim 1, wherein the illumination procedure is performed with a radiation in a visible spectrum.

4. The method of claim 1, further comprising receiving diffuse reflectance spectra based on the illumination procedure, wherein the at least one characteristic is determined based on the received diffuse reflectance spectra.

5. The method of claim 4, further comprising inverting the diffuse reflectance spectra using an inverse Monte Carlo procedure.

6. The method of claim 5, further comprising determining a concentration of at least one of (i) an oxy-myoglobin, (ii) a deoxy-myoglobin or (iii) a met-myoglobin based on the inverted diffuse reflectance spectra.

7. The method of claim 5, further comprising:
determining a concentration of a met-myoglobin based on the inverted diffuse reflectance spectra; and
performing at least one of (i) an analysis of variance test or (ii) a Tukey's multiple comparison test on the met-myoglobin concentration.

8. The method of claim 5, further comprising fitting the inverted diffuse reflectance spectra to a wavelength dependent model.

9. The method of claim 8, further comprising receiving a plurality of coefficients based on results of the fitting step, wherein the at least one characteristic is determined based on the coefficients.

10. The method of claim 1, wherein the at least one characteristic includes a classification of the tissue.

11. The method of claim 10, wherein the classification is regarding the at least one tissue having a lesion thereon.

12. The method of claim 1, further comprising repeating the ablation and illumination procedures until a permanent lesion is formed on the at least one tissue.

13. The method of claim 1, further comprising determining a baseline diffuse reflectance spectra associated with the at least one tissue before the ablation procedure.

14. The method of claim 1, further comprising flushing the at least one tissue.

15. The method of claim 1, further comprising electrically mapping a surface of the at least on tissue.

16. A system for determining at least one characteristic of at least one tissue, comprising:
a computer hardware arrangement configured to:
ablate the at least one tissue,
illuminate the at least one tissue during the ablation procedure, and
determine the at least one characteristic based on the ablation and illumination procedures.

17-30. (canceled)

31. A non-transitory computer-accessible medium having stored thereon computer-executable instructions for determining at least one characteristic of at least one tissue, wherein, when a computer arrangement executes the instructions, the computer arrangement is configured to perform procedures comprising:
ablating the at least one tissue;
illuminating the at least one tissue during the ablation procedure; and
determining the at least one characteristic based on the ablation and illumination procedures.

32-45. (canceled)

46. A system for determining at least one characteristic of at least one tissue, comprising
a first electromagnetic radiation source configured to (i) generate at least one first radiation and (ii) provide the at least one first radiation to the at least one tissue so as to partially ablate the at least one tissue;
a second electromagnetic radiation source configured to (i) generate at least one second radiation, and (ii) provide the at least one second radiation to the at least one tissue;
a detector arrangement configured to (i) obtain a return radiation from the at least one tissue that is based on the at least one second radiation impacting the at least one tissue and the at least partial ablation caused by the at least one first radiation, and (ii) provide data associated with at least one further characteristic of the returned radiation; and
a computer processing arrangement configured to determine the at least one characteristic based on the data.

47-50. (canceled)

51. The system of claim 46, further comprising at least one flushing arrangement configured to flush the at least one tissue.

52. The system of claim 46, further comprising a voltage arrangement configured to generate at least one voltage, wherein the detector arrangement is further configured to obtain a return voltage from the at least one tissue that is based on the at least one second radiation impacting the at least one tissue.

53. The system of claim 52, wherein the computer processing arrangement is further configured to generate at least one map of a surface of the at least one tissue based on the return voltage.

54. A method for ablating at least one tissue, comprising:
determining at least one location of at least one dead portion of the at least one tissue;
providing the at least one location to an ablative source arrangement;
moving the ablative source arrangement to at least one further location based on the at least one location; and
ablating the at least one further location of the at least one tissue.

55. The method of claim 54, wherein the determination procedure is based on at least one intensity and at least one wavelength of a radiation produced by the ablative source arrangement.
